(19) United States
(12) Patent Application Publication
(10) Pub. No.: US 2009/0075902 A1
(43) Pub. Date: Mar. 19, 2009

(54) INHIBITING THE SIGNS OF AGING BY INHIBITING NF-KAPPA B ACTIVATION

(76) Inventors: Paul D. Robbins, Mt. Lebanon, PA (US); Laura J. Niedernhofer, Pittsburgh, PA (US)

Correspondence Address: BAKER BOTTS L.L.P., 30 ROCKEFELLER PLAZA, 44TH FLOOR, NEW YORK, NY, 10112-4498, US

(21) Appl. No.: 12/126,634

(22) Filed: May 23, 2008

Related U.S. Application Data
(60) Provisional application No. 60/940,312, filed on May 25, 2007.

(51) Int. Cl. A61K 38/04; A61K 31/277; A61K 31/44; A61K 38/02; C07K 14/00; C07K 7/08

(52) U.S. Cl. 514/15; 514/520; 514/277; 514/2; 530/327; 530/326

(57) ABSTRACT

The present invention relates to methods and compositions for reducing and/or delaying one or more signs of aging which comprise inhibiting NF-kappa B activation. It is based, at least in part, on the discovery that a peptide which inhibits IKK-β interaction with NEMO, linked to a transducing peptide, inhibits the development of various indicia of senescence in a murine model of aging, Ercc1^{-/Δ} mice.

### PRIORITY CLAIM

This application claims priority to U.S. Provisional Application No. 60/940,312 filed May 25, 2007, the contents of which is incorporated in its entirety herein.

### GRANT INFORMATION

Not applicable.

### 1. INTRODUCTION

The present invention relates to methods for reducing and/or delaying one or more signs of aging which comprise inhibiting NF-kappa B activation, preferably by blocking the interaction between NF-κB essential modulator (“NEMO”) with IκB kinase-β (IKK-β) at the NEMO binding domain (NBD).

### 2. BACKGROUND OF THE INVENTION

### 2.1 Aging

It is estimated that in the next 25 years, the number of individuals over the age of 65 in the United States will double (U.S. Department of Health and Human Services, 2003). With increased chronological age, there is progressive attrition of homeostatic reserve of all organ systems (Resnick and Dosa, 2004). As a consequence, aged individuals have a dramatically increased risk of numerous debilitating diseases including bone fractures, cardiovascular disease, cognitive impairment, diabetes and cancer (Resnick and Dosa, 2004). Therefore, as American demographics shift, increasing demands are placed on our health care system (Crippen, 2000). Identifying strategies to prevent or delay age-associated frailty and diseases is imperative for maintaining the health of our population as well as our nation's economy.

The molecular basis of the progressive loss of homeostatic reserve with aging is controversial (Kirkwood, 2005b; Resnick and Dosa, 2004). There is strong evidence that genetics contribute significantly to lifespan and end-of-life fitness (Hekimi and Guarente, 2003). This was demonstrated by identifying single genes that when mutated or overexpressed attenuate and extend lifespan, respectively (Kurosu et al., 2005). Many of the genes that regulate lifespan affect the growth hormone (GH)/insulin-like growth factor 1 (IGF1) axis, which controls cellular proliferation and growth (Kenyon, 2005). Suppression of this axis extends lifespan significantly and delays age-related diseases (Bartke, 2005).

Alternatively, the disposable soma theory of aging posits that aging is the consequence of accumulation of stochastic molecular and cellular damage (Kirkwood, 2005b). The precise nature of the damage that is responsible for aging-related degenerative changes remains ill-defined, but may include mitochondrial damage, telomere attrition, nuclear dysmorphology, accumulation of genetic mutations, DNA, protein or membrane damage.

There are several lines of evidence to support the notion that DNA damage is one type of molecular damage that contributes to aging. At the forefront of this is the observation that the majority of human progerias (or syndromes of accelerated aging) are caused by inherited mutations in genes required for genome maintenance, including Werner syndrome, Cockayne syndrome, trichothiodystrophy and ataxia telangiectasia (Hasty et al., 2003). Furthermore both DNA lesions (Hamilton et al., 2001) and genetic mutations caused by DNA damage (Dolle et al., 2002) accumulate in tissues with aging. Finally, mice harboring germ-line mutations that confer resistance to genotoxic stress are long-lived (Maier et al., 2004; Migliaccio et al., 1999).

ERCC1-XPF is a highly conserved structure-specific endonuclease that is required for at least two DNA repair mechanisms in mammalian cells: nucleotide excision repair (Sijbers et al., 1996) and DNA interstrand crosslink repair (Niedernhofer et al., 2004). Genetic deletion of either Ercc1 or Xpf in the mouse causes an identical and very severe phenotype (McWhir et al., 1993; Tian et al., 2004; Weeda et al., 1997). Embryonic development of null mice is normal, but postnatally they develop numerous symptoms associated with advanced age including epidermal atrophy and hyperpigmentation, visual impairment, cerebral atrophy with cognitive deficits, cerebellar degeneration, hypertension, renal insufficiency, decreased liver function, anemia and bone marrow degeneration, osteopenia, sarcopenia, cachexia, and decreased lifespan (Niedernhofer et al., 2006; Prasher et al., 2005; Weeda et al., 1997, and see International Patent Application Publication No. WO2006/052136).

To determine if this progeroid phenotype had commonalities with the natural aging process, the transcriptome from the liver of Ercc1−/− mice was compared to that of old wild type (wt) mice and a highly significant correlation was identified (Niedemhofer et al., 2006). Similar expression changes were also identified in young wt mice after chronic exposure to a DNA damaging agent. This provides direct experimental evidence that DNA damage induces changes that mimic aging at the fundamental level of gene expression.

Gene ontology classification of the expression data was used to predict pathophysiologic changes that were similar in Ercc1−/− mice and old wild type mice (Niedernhofer et al., 2006). These predictions were tested comparing Ercc1−/− mice, young and old wt mice. For all predictions tested, Ercc1−/− were more similar to old mice than to their wt littermates despite the vast difference in age (3 wks vs. 120 wks). Both Ercc1−/− and old mice displayed hyposomatotropism, hepatic accumulation of glycogen and triglycerides, decreased bone density, increased peroxisome biogenesis, increased apoptosis and decreased cellular proliferation. Therefore, Ercc1−/− and old mice share not only broad changes in gene expression, but also endocrine, metabolic and cell signaling changes. This implies that ERCC1-deficient mice are an accurate and rapid model system for studying systemic aging in mammals. A case of human progeria caused by ERCC1-XPF deficiency with symptoms near-identical to those observed in ERCC1-deficient mice has been reported (Niedernhofer et al., 2006). Therefore function of ERCC1-XPF is conserved from man to mouse and the discovery of what is driving aging-like degenerative changes in ERCC1-deficient mice will have direct implications for human health.

### 2.2 NF-κB

NF-κB is a highly conserved, ubiquitously expressed family of transcription factors. NF-κB is a key regulator of cell survival and proliferation in response to a numerous types of stress including oxidative, genotoxic, mitogenic and inflammatory (Hu and Hung, 2005). NF-κB family members include p65, c-Rel, Rel-B, p50 and p52. NF-κB exists as a dimer, with the most common being p50-p65. In the absence of stress, NF-κB is retained in the cytoplasm through its association with IκB proteins. IκB proteins bind to NF-κB and mask their nuclear localization signals. Cellular stress leads to phosphorylation of IκB proteins at specific N-terminal serine residues, by IκB kinase (IKK). Phosphorylated IκB proteins are polyubiquitinated and thereby targeted for degradation by the 26S proteosome. This releases NF-κB, which then translocates to the nucleus, binds to NF-κB cognate sequences in enhancer elements of target genes and induces transcription.

Activation of NF-κB is implicated in numerous pathophysiologic conditions including chronic inflammatory diseases (rheumatoid arthritis, asthma and inflammatory bowel disease) (Tak and Firestein, 2001) and cancer (Pikarsky et al., 2004). NF-κB is also activated in a variety of tissues of aged rodents relative to young animals, including the skin, liver, kidney, cerebellum, cardiac muscle and gastric mucosa (Bregegere et al., 2006; Giardina and Hubbard, 2002; Helenius et al., 1996a; Helenius et al., 1996b; Korhonen et al., 1997; Xiao and Majumdar, 2000). Furthermore, NF-κB activation has been implicated in driving cellular senescence (Gosselin and Abbadie, 2003). There are several reports of NF-κB inhibitors modulating molecular effects associated with aging (Kim et al., 2006; Go et al, 2005).

One strategy for inhibiting NF-κB activation involves inhibiting phosphorylation of IκB proteins, thereby preventing mobilization of NF-κB into the nucleus. A variety of inflammatory signals stimulate the NF-kB pathway by activating the IkB kinase (IKK) complex, comprised of three subunits including the IKKgamma, also called NEMO for “NF-κB essential modulator”, regulatory subunit and two catalytic subunits, IKKalpha and beta. The interaction of these subunits allows for phosphorylation of IKK, resulting in activation of kinase activity and subsequent phosphorylation IκB, the cytoplasmic inhibitor of NF-kB. After phosphorylation, IkB is subsequently degraded which releases NF-κB and reveals a nuclear localization signal, allowing NF-κB to shuttle to the nucleus and activate pro-inflammatory genes.

A short peptide (TALDWSWLQTE; SEQ ID NO:1) derived from the Nemo Binding Domain (NBD) of IKKβ blocks association of NEMO with IKKbeta, preventing NF-κB activation (di Meglio et al., 2005; May et al., 2000). Importantly, the NBD peptide does not affect basal activity of NF-κB, which is required for mouse development and tissue homeostasis (Li et al., 1999; Nenci et al., 2007; Rudolph et al., 2000; Tanaka et al., 1999), but only suppresses induction of NF-κB activity in response to inflammatory stimuli and certain types of stress (May et al., 2000). Systemic administration of NBD peptide is not toxic to mice and inhibition of NF-κB by this peptide correlates with therapeutic response in numerous models of chronic inflammation (Dai et al., 2004; Dasgupta et al., 2004; Dave et al., 2006; di Meglio et al., 2005; Jimi et al., 2004). The use of peptides to block this interaction is disclosed in U.S. Pat. Nos. 6,864,355 and 7,049,395 and United States Patent Application Publication No. 2006/0293244. Another inhibitor of NF-κB that has been identified is a small molecule inhibitor of IKK-β, referred to as “Compound A,” which is a 2-amino-3-cyna-4-alkyl-6-(2-hydroxyphenyl)pyridine derivative.

### 3. SUMMARY OF THE INVENTION

The present invention relates to methods for reducing and/or delaying one or more signs of aging which comprise inhibiting NF-κB activation. It is based, at least in part, on the discovery that a peptide which inhibits IKK-β interaction with NEMO, linked to a transducing peptide, inhibits the development of various indicia of senescence in a murine model of aging, Ercc1^{-/Δ} mice.

The present invention further provides for compositions comprising a NF-κB activation inhibitor which is a NBD peptide comprising a polylysine transduction peptide. In non-limiting embodiments, such compositions may be used according to the methods of the invention to reduce and/or delay one or more signs of aging.

### 4. BRIEF DESCRIPTION OF THE FIGURES

FIG. 1. Schematic diagram of mErcc1 targeting constructs. In the knock-out allele (−) exon 7 is interrupted with a neo cassette. In the hypomorphic allele (Δ), a small deletion was placed at the very C-terminus of the protein to mimic human ERCC1 and a neo cassette was placed in intron 9. In the knock-down allele (↓), exon 7 was fused with the cDNA of exon 8-10 and the entire region floxed with loxP sites recognized by Cre-recombinase. The neomycin cassette situated in the 3′UTR disrupts Ercc1 expression, if not removed by Cre recombinase. The level of Ercc1 mRNA expressed from each allele relative to the wt allele is indicated.

FIG. 2. Immunodetection of XPF in protein extracts isolated from the liver of Ercc1 mutant mice. The genotype of the mice is indicated above the lanes. Below the blots are indicated the calculated level of XPF expression in the Ercc1 mutant mice, relative to wild type (100%).

FIG. 3. Clonogenic survival assay measuring the sensitivity of ERCC1-deficient cells to the DNA damaging agent mitomycin C. Wild-type data is represented by a circle; Ercc1^{-/↓} by a square; Ercc1^{-/Δ} by a triangle, and Ercc1^{-/-} by an open square.

FIG. 4. Lifespan of mice expressing various levels of ERCC1-XPF compared to wt C57B1/6 mice (100%; adapted from (Rowlatt et al., 1976)). Wild-ype data is represented by a diamond; Ercc1^{-/↓} by a square; and Ercc1^{-/Δ} by a triangle.

FIG. 5. Up-regulation of NF-κB in aged wt and progeroid Ercc1^{-/Δ} mouse liver relative to young wt mice. Sections of cryopreserved liver were stained with Dapi to identify nuclei (blue), actin to highlight the cytoplasm (red) and for the p65 subunit of NF-κB (green). [this figure is in black and white]

FIG. 6. Exposure paradigm for pilot study to determine the impact of 8K-NBD on quality of life in progeroid Ercc1^{-/Δ} mice. 8K-NBD was dissolved in 5% DMSO in PBS and injected intraperitoneally. A littermate Ercc1^{-/Δ} mouse was treated with an equal volume of vehicle only (PBS). The study was concluded at 19 wks when the mouse treated with vehicle only became profoundly ataxic and cachectic, requiring euthanization.

FIG. 7A-B. A. Images of sibling Ercc1^{-/Δ} mice, one chronically treated with 8K-NBD, the other vehicle only. The mice treated with vehicle only had early onset priapism (arrow), loss of vision, and were incontinent (*). In addition, dystonia (dotted arrow), kyphosis, ambulation and general appearance were improved in the mouse treated with 8K-NBD. B. Summary Table.

FIG. 8. 8K-NBD delays weight loss in progeroid Ercc1^{-/Δ} mice. A mouse systemically treated with 8K-NBD (diamonds) maintained body weight significantly longer than untreated mice (circles) or a mouse treated with vehicle only (triangles).

FIG. 9A-B. A. Mice were tested for their ability to cross a 24″ balance beam weekly and scored from 5 (able to cross) to 1 (fall off beam). A mouse treated with 8K-NBD (diamonds) performed better than untreated (circles) mice (n=7) (mice treated with vehicle+triangles). B. Mice were suspended from a wire cage top and the time that they were able to remain suspended recorded weekly. Untreated mice (circles, n=7) and a mouse treated with vehicle only (triangles) displayed a more precipitous decline in grip strength with age then a mouse treated with 8K-NBD (diamonds).

FIG. 10. Exposure paradigm for pilot study to determine the impact of 8K-NBD on histopathologic changes in progeroid Ercc1^{-/Δ} mice. 8K-NBD was dissolved in 5% DMSO in PBS and injected i.p.

FIG. 11A-B. A. Paraffin-embedded liver sections of liver. Centrilobular necrosis and large polyploid nuclei characteristic of Ercc1^{-/Δ} mice were observed in the mouse treated with vehicle only. Mice treated with 8K-NBD exhibit loss of hepatic architecture, consistent with liver regeneration, and a greater cytoplasm/nuclear ratio in hepatocytes. B. Paraffin-embedded sections of skeletal muscle from a mouse treated with vehicle only display evidence of necrosis (arrows). In contrast, SKM from a mouse treated with 8K-NBD reveals no pathologic changes.

FIG. 12. Treatment schedule for third series of experiments; treatment with 8K-NBD or negative control 8k-mNBD.

FIG. 13. Table showing phenotypic changes associated with aging in Ercc1^{-/Δ} mice treated with 8K-NBD or negative control 8K-mNBD.

FIG. 14A-D. Comparison of levels of neurodegeneration, as determined by glial fibrillary acidic protein (GFAP) staining, in (A) a young wild-type mouse; (B) a 2-year old (aged) wild-type mouse; (C) a Ercc1^{-/Δ} mouse treated with 8K-NBD according to the regimen of FIG. 12; and (D) a Ercc1^{-/Δ} mouse treated with negative control 8K-mNBD.

FIG. 15A-D. Comparison of levels of hepatocellular senescence, as measured by staining for p16 protein, in (A) a young wild-type mouse; (B) a 2-year old (aged) wild-type mouse; (C) a Ercc1^{-/Δ} mouse treated with 8K-NBD according to the regimen of FIG. 12; and (D) a Ercc1^{-/Δ} mouse treated with negative control 8K-mNBD.

FIG. 16A-F. Comparison of osteoporotic changes in Ercc1^{-/Δ} mice treated with 8K-NBD or negative control 8K-mNBD. Gross macroscopic and cross-sectional views from micro-CT are shown. (A) gross macroscopic view of vertebra of wild-type, untreated 17 week old mouse; (B) cross-sectional view of vertebra of wild-type untreated mouse shown in (A); (C) gross macroscopic view of vertebra of sibling Ercc1^{-/Δ} mouse treated with 8K-mNBD; (D) cross-sectional view of vertebra of Ercc1^{-/Δ} mouse of (C); (E) gross macroscopic view of vertebra of sibling Ercc1^{-/Δ} mouse treated with 8K-NBD; (F) cross-sectional view of vertebra of Ercc1^{-/Δ} mouse of (E).

FIG. 17. Family tree to create genetic depletion of NF-κB through crossing a p65^{+/-} Ercc^{+/-} parent with a Ercc^{+/-} parent and producing p65^{+/-} Ercc^{-/-} f1 progeny.

FIG. 18. Survival of Ercc^{-/-} mice (diamonds) compared with a p65^{+/-} Ercc^{-/-} mice (squares).

FIG. 19A-D. Fluorescently labeled protein transduction domains (“PTDs”) penetrate mouse skin. A. The PTD is 6-His (His-His-His-His-His-His (SEQ ID NO: 3)); B. The PTD is 6-R (Arg-Arg-Arg-Arg-Arg-Arg (SEQ ID NO:4)); C. The PTD is 8K (Lys-Lys-Lys-Lys-Lys-Lys-Lys-Lys (SEQ ID NO:5)); D. The PTD is Con-P (Ala-Arg-Phe-Leu-Glu-His-Gly-Ser-Asp-Lys-Ala-Thr (SEQ ID NO:6)).

FIG. 20. Transduction of mouse skin by 8K.

FIG. 21. Transduction of mouse skin by peptides with or without cream.

### 5. DETAILED DESCRIPTION OF THE INVENTION

For clarity of presentation, and not by way of limitation, the detailed description of the invention is divided into the following subsections:

(i) inhibitors of NF-κB activation;

(ii) signs of aging that may be modulated; and

(iii) methods of treatment.

### 5.1 Inhibitors of NF-κB Activation

The present invention provides for the use of molecules which inhibit NF-κB activation. “NF-κB activation” means the process whereby NF-κB is freed from its cytoplasmic complex, enters the nucleus, binds its cognate regulatory elements and enhances gene transcription. Important aspects of NF-κB activation include (i) binding of the regulatory subunit named “NF-κB essential modulator” (NEMO or IKKγ) to IKKβ and IKKalpha, activating the IKK complex (ii) phosphorylation of IκB, (iii) transport of NF-κB through the nuclear membrane, (iv) DNA binding of NF-κB, and (v) transactivation by NF-κB. An inhibitor of NF-κB activation may inhibit activation of any of these five aspects.

Inhibitors of NEMO binding include peptides comprising a NEMO binding domain “NBD”, referred to herein as “NBD peptides”. Examples of such peptides, which may further comprise a protein transduction domain, include peptides disclosed in U.S. Pat. No. 6,864,355, U.S. Pat. No. 7,049,395, and/or United States Patent Application Publication No. US 2006/0293244 (each of which is incorporated by reference herein in its entirety).

Examples of transduction domain-containing peptides which may be comprised in NBD peptides for use according to the invention include transduction domain-containing peptides disclosed in U.S. Pat. No. 6,881,825, International Patent Application No. PCT/US03/04632, United States Patent Application Publication No. 2003-0104622 A1, United States Patent Application Publication No. 2003-0219826 A1, and United States Patent Application Publication No. 2005-0074884 A1 (each of which is incorporated by reference herein in its entirety). In particular, non-limiting embodiments of the invention, the transduction domain-containing peptide is a polylysine of between four and twelve lysine residues, preferably eight lysine residues. Other examples of transduction domain-containing peptides are 6-His, 6-R, and Con-P.

Optionally, a spacer peptide of between 1 and 10 amino acid residues may be included between the NBD and the transduction domain-containing peptide.

In specific, non-limiting embodiments of the invention, the NBD peptide comprises (i) a polylysine transduction peptide of between four and twelve lysine residues, preferably eight lysine residues; and (ii) a NEMO binding domain with a sequence which is either (a) TALDWSWLQTE (SEQ ID NO:1) or (b) a sequence which is at least 90 percent homologous to SEQ ID NO:1 or (c) a 9-11 amino acid sequence which differs from SEQ ID NO:1 in no more than two amino acids or (d) a 8-11 amino acid sequence which differs from SEQ ID NO:1 in no more than three amino acids. According to such embodiments, the NBD peptide may be between about 12 and 50 amino acids in length, or between about 12 and 30 amino acids in length, or between about 12 and 20 amino acids in length. Amino acid sequences which neither function in transduction or NEMO binding may, without limitation, be functionally essentially silent or may, for example, improve the stability of the peptide. In one specific, non-limiting embodiment, the NBD peptide may be KKKKKKKK-GG-TALDWSWLQTE (SEQ ID NO:2); said peptide may be comprised in a pharmaceutical composition, optionally further comprising a suitable pharmaceutical carrier.

In a second set of non-limiting embodiments, the inhibitor of NF-κB activation may be a 2-amino-3-cyna-4-alkyl-6-(2-hydroxyphenyl)pyridine derivative such as, but not limited to, Compound A (Murata et al., 2003; Mustafa and Leverve, 2001; Ziegelbauer et al., 2005). Compound A (CpdA), 7-[2-(cyclopropylmethoxy)-6-hydroxyphenyl]-5-[(3S-3-piperidinyl]-1,4-dihydro-2H-pyrido[2,3-d][1,3]oxazin-2-one hydrochloride, a 2-amino-3-cyano-4-alkyl-6-(2-hydroxyphenyl)pyridine derivative, is optionally present in “Compound B”, an orally active enantiomeric mixture of Compound A (Ziegelbauer et al, 2005) which may also be used according to the invention.

In a third set of non-limiting embodiments, the inhibitor of NF-κB activation may be BAY11-7082 or BAY11-7085 (Axxora L.L.C., San Diego, Calif.) (Petegnief et al., 2001, Neuroscience 104:223).

In a fourth set of non-limiting embodiments, the inhibitor of NF-κB activation may be SC-514 (Kishore et al., 2003, J. Biol. Chem./278(35):32861.

In a fifth set of non-limiting embodiments, the inhibitor of NF-κB activation may be MG132 (Calbiochem, La Jolla, Calif.).

In a sixth set of non-limiting embodiments, the inhibitor of NF-κB activation may be tosyl-Phe-chloromethylketone (“TPCK”).

In a seventh set of non-limiting embodiments, the inhibitor of NF-κB activation may be (N-6-chloro-7-methoxy-9H-β-carbolin-8-yl)-2-methylnicotinamide (ML120B, Wen et al., 2006, J. Pharm. Exp. Ther. 317:989-1001.

In an eighth set of non-limiting embodiments, the inhibition of NF-κB activation may be Celastrol (Lee, et al., 2006, Biochem. Pharmacol. 72(10): 1311-1321).

In a ninth set of non-limiting embodiments, the inhibition of NF-κB activation may be PG201 (Shin, et al., 2005, Biochem. Biophys. Res. Common. 331(4): 1469-1477).

In a tenth set of non-limiting embodiments, the inhibition of NF-κB activation may be MLN0415 (Millenium Pharmaceuticals, Cambridge, Mass.).

### 5.2 Signs of Aging that May be Modulated

The present invention may be used to inhibit the development or progression of one or more signs of aging, including, but not limited to, epidermal atrophy, epidermal hyperpigmentation, rhytid (wrinkles), photoaging of the skin, hearing loss, visual impairment, cerebral atrophy, cognitive deficits, trembling, ataxia, cerebellar degeneration, hypertension, renal insufficiency, renal acidosis, incontinence, decreased liver function, hypoalbuminemia, hepatic accumulation of glycogen and triglycerides, anemia, bone marrow degeneration, osteopenia, kyphosis, degenerative joint disease, intervertebral disc degeneration, sarcopenia, muscle weakness, dystonia, increased peroxisome biogenesis, increased apoptosis, decreased cellular proliferation, cachexia, and decreased lifespan. “Inhibiting the development” of a sign of aging means delaying the onset, slowing the progression, or reducing the manifestation, of a sign of aging.

The present invention may be used to improve age-related performance in a geriatric subject. “Improving performance” refers to any aspect of performance, including cognitive performance or physical performance, such as, but not limited to, the ability to be self-sufficient, to take care of (some but not necessarily all) personal needs, to be ambulatory or otherwise mobile, or interaction with others.

The present invention may be used to prolong survival of a geriatric subject, for example, relative to an age-matched, clinically comparable control not treated according to the invention.

### 5.3 Methods of Treatment

Accordingly, in one set of embodiments, the present invention provides for a method of inhibiting one or more signs of aging in a subject in need of such treatment, comprising administering, to the subject, an effective amount of an inhibitor of NF-κB activation.

In a related set of embodiments, the present invention provides for a method of improving age-related performance in a geriatric subject, comprising administering to a subject an effective amount of an inhibitor of NF-κB activation.

In another related set of embodiments, the present invention provides for a method of prolonging survival of a geriatric subject, comprising administering, to the s subject, an effective amount of an inhibitor of NF-κB activation.

The inhibitor of NF-κB activation may be administered systemically to achieve distribution throughout the body or may be administered to achieve a local effect. The route of administration may be selected depending on the intended effect. As non-limiting examples, systemic administration, to achieve therapeutic levels throughout the body, may be achieved using an inhibitor suitable for distribution throughout the body, administered via any standard route, including but not limited to oral, intravenous, inhalation, subcutaneous, or intramuscular routes. Non-limiting examples of local administration include, but are not limited to, intrathecal administration to treat central nervous system manifestations of aging, ocular instillation to treat visual disturbances, intramuscular injection may be used to treat muscle wasting, topical administration to prevent or reverse skin aging etc.

Topical formulations may include administering the NF-κB activation inhibitor, optionally comprised in microsphere, microcapsule, or liposome, in a cream, lotion, organic solvent, or aqueous solution.

Inhibitors according to the invention may be administered in a suitable pharmaceutical carrier (e.g. sterile water, normal saline, phosphate buffered saline, etc.). Not by way of limitation, inhibitors may be administered as a solution, as a suspension, in solid form, in a sustained release formulation, in a topical cream formulation, etc. In particular non-limiting examples, an inhibitor may be incorporated into a microcapsule, nanoparticle or liposome.

An effective dose may be calculated by determining the amount needed to be administered to produce a concentration sufficient to achieve the desired effect in the tissue to be treated, taking into account, for example, route of administration, bioavailability, half-life, and the concentration which achieves the desired effect in vitro or in an animal model system, using techniques known in the art.

Non-limiting examples of doses of NBD peptide inhibitors include between 0.1 and 50 mg/kg, or between 1 and 25 mg/kg, or between 2 and 20 mg/kg, or about 2 mg/kg, or about 10 mg/kg, which may be administered daily, at least 5 times a week, at least 3 times a week, at least twice a week, at least once a week, at least twice a month, at least once a month, at least once every three months, or at least once every six months.

In particular, non-limiting embodiments, a subject may be treated with a NBD peptide inhibitor using a regimen comprising a loading period followed by a maintenance period, wherein the loading period includes treatment, with 1-20 mg/kg or 2-10 mg/kg, daily or every other day for a period of 5-10 days, followed by a maintenance period which includes 1-10 mg/kg, or 10-50 mg/kg, given once a week, twice a week, three times a week, every other week, or once a month.

In other non-limiting embodiments:

where Compound A is the inhibitor, the dose may be between about 0.1 and 20 mg/kg, or between about 0.3 and 10 mg/kg, or between about 2 and 8 mg/kg, or about 5 mg/kg;

where Compound B is the inhibitor, the dose may be between about 0.1 and 40 mg/kg, or between about 0.3 and 20 mg/kg.

where BAY11-7082 or BAY11-7085 is the inhibitor, the dose may be between about 0.1 and 10 mg/kg or between about 1 and 10 mg/kg or about 5 mg/kg;

where SC-514 is the inhibitor, the dose may be such that the resulting local concentration at the site of treatment is up to 100 μM for 100% inhibition of NF-κB, as the IC₅₀ is 11.2 μM or 8-20 μM (Kishori 2003);

where MG132 is the inhibitor, the dose may be such that the resulting local concentration at the site of treatment is between about 0.1 and 0.5 μM;

where TPCK is the inhibitor, the dose may be such that the resulting local concentration at the site of treatment is between about 5-10 μM;

where ML120B is the inhibitor, the dose may be such that the resulting local concentration at the site of treatment is up to 20 μM (Wen 2006), as the IC₅₀ is 60 nM;

where Celastrol is the inhibitor, the dose may be between about 1 and 20 mg/kg or between about 10 mg/kg and 30 mg/kg (Lee, 2006) or

where PG201 is the inhibitor, the dose may be about 200 mg/kg (Park, 2005);

and in any of the foregoing, the dose may be administered daily, at least 5 times a week, at least 3 times a week, at least twice a week, at least once a week, at least twice a month, at least once a month, at least once every three months, or at least once every six months.

### 6. EXAMPLE

### A Murine Model of Aging

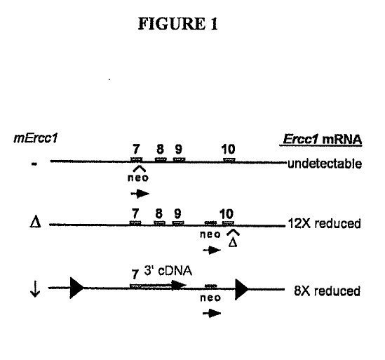

If the capacity to repair stochastic molecular damage is an important determinant of lifespan, then the prediction is that organisms with reduced capacity for repair would have proportionally reduced lifespan. To test this, a series of mice were generated expressing various levels of ERCC1-XPF DNA repair endonuclease and therefore different capacities for DNA repair and lifespan. Two new mErcc1 targeting constructs were cloned (FIG. 1). One construct (Δ) contains a deletion of the last 7 amino acids of ERCC1 to humanize the protein and a neomycin cassette in intron 9 (Weeda et al., 1997). In the second construct (↓), genomic sequence of Ercc1 exon 7 was fused to a cDNA encoding exons 8-10 and the fusion floxed with loxP sites. A neomycin cassette was inserted in the 3′UTR.

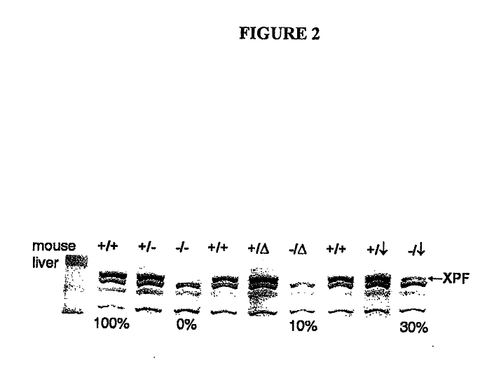

Compound heterozygote mice were bred combining each of these new alleles with the null allele (Ercc1^{-/Δ} and Ercc1^{-/↓} mice) and liver isolated for extraction of RNA and protein. Ercc1 mRNA was measured by qRT-PCR. In both Ercc1^{-/Δ} and Ercc1^{-/↓} mice, expression of Ercc1 is reduced (12-fold and 8-fold respectively) due to transcriptional interference by the neomycin cassette (Lantinga-van Leeuwen et al., 2004). Direct detection of ERCC1 protein is not possible due to the lack of an antibody that recognizes the murine protein. As a surrogate, the essential binding partner of ERCC1, XPF, was measured in mouse liver (FIG. 2). XPF levels in Ercc1^{-/Δ} mice were 10% that of wt mice, whereas Ercc1^{-/↓} mice were found to have 30% of the normal level of XPF.

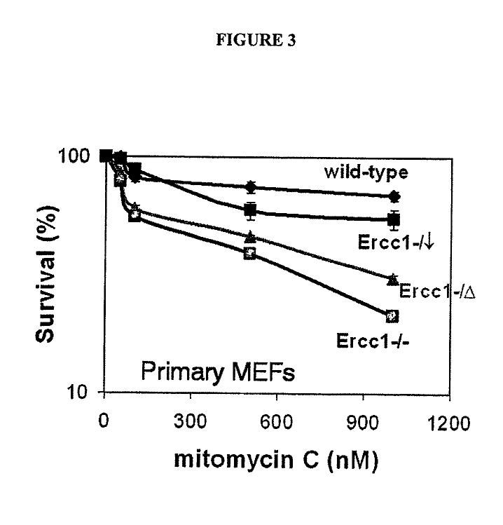

As a consequence, primary mouse embryonic fibroblasts (MEFs) isolated from both mouse strains are proportionately more sensitive to DNA damaging agents than wt cells, but less sensitive than ERCC1-null cells (FIG. 3). Therefore by titrating the endogenous level of ERCC1-XPF, we have four mouse strains (wt, Ercc1^{-/Δ}, Ercc1^{-/↓}, and Ercc1−/−) with different capacities for DNA repair.

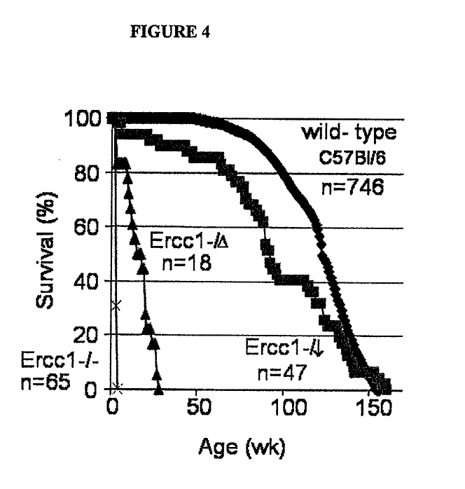

The maximum lifespan of each mouse strain was measured and compared to wt C57B1/6 mice (FIG. 4). Ercc1^{-/-} mice were found to have, at maximum, a lifespan of 1 month. Ercc1^{-/Δ} mice had a maximum lifespan of 7 months. Ercc1^{-/↓} mice had a normal maximum lifespan. However their median lifespan was significantly reduced compared to wt mice [90 vs. 130 wks, respectively (Rowlatt et al., 1976)]. This demonstrates that capacity for molecular repair, in particular DNA repair, is a direct determinant of lifespan.

Importantly, Ercc1^{-/Δ} mice develop the same premature aging symptoms as Ercc1−/− mice (Niedernhofer et al., 2006). However the age at onset of the symptoms is delayed; Ercc1^{-/Δ} mice are asymptomatic for the first 8 wks of life. Beginning at 8 wks, the mice begin to display a constellation of progressive symptoms associated with advanced age including: trembling, ataxia, cerebral atrophy, renal acidosis, decreased liver function, hypoalbuminemia, bone marrow degeneration, osteopenia, kyphosis, dystonia, muscle wasting, growth retardation, cachexia and loss of vision. The phenotype of the mice is remarkably homogenous with all mice displaying an identical spectrum of symptoms in a highly predictable order of appearance. This indicates that the Ercc1^{-/Δ} strain is a uniquely accurate and rapid model system for studying mammalian aging and many of the debilitating symptoms associated with human aging.

### 7. EXAMPLE

### NF-κB is Upregulated in Liver in Old Wild-Type and progeroid ERCC1^{-/Δ} mice

NF-κB transcription factor is activated in a variety of tissues of aged rodents relative to young animals including the liver, kidney, cerebellum, skin, cardiac muscle and gastric mucosa (Bregegere et al., 2006; Giardina and Hubbard, 2002; Helenius et al., 1996a; Helenius et al., 1996b; Korhonen et al., 1997; Xiao and Majumdar, 2000). In liver, levels of the p65 subunit of NF-κB are elevated (Helenius et al., 2001). Activation of NF-κB has been implicated in driving cellular senescence (Gosselin and Abbadie, 2003). Thus, this transcription factor may play a direct role in driving aging and its associated degenerative processes.

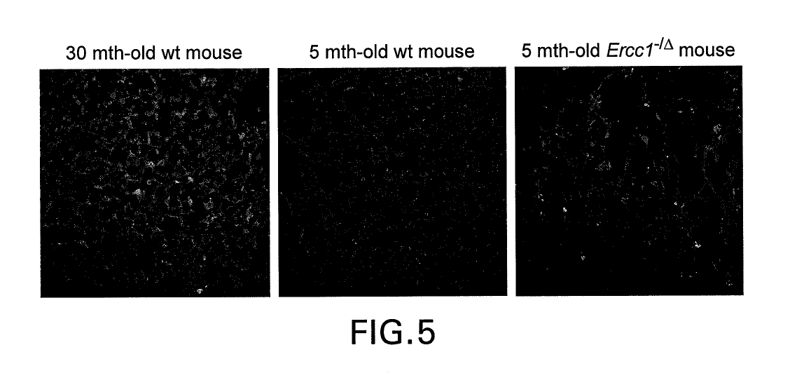

Hepatocytes from Ercc1^{-/-} and Ercc1^{-/Δ} mice show numerous characteristics of senescence including large polyploid nuclei, intranuclear inclusions and intracellular lipid deposition (Niedernhofer et al., 2006). To determine whether p65 levels were increased in liver of progeroid Ercc1^{-/Δ} mice (FIG. 5), immunohistological studies were performed. Immunodetection of p65 in liver sections revealed increased protein in both aged wt and 5 month-old Ercc1^{-/Δ} mouse liver compared to 5 month-old wt mice. This establishes another parallel between the progeria of Ercc1^{-/Δ} mice and natural aging and indicates that NF-κB levels are abnormally elevated in Ercc1^{-/Δ} mice.

### 8. EXAMPLE

### 8K-NBD Delays and Ameliorates Aging-Like Symptoms and pathology in ERCC1^{-/Δ} mice

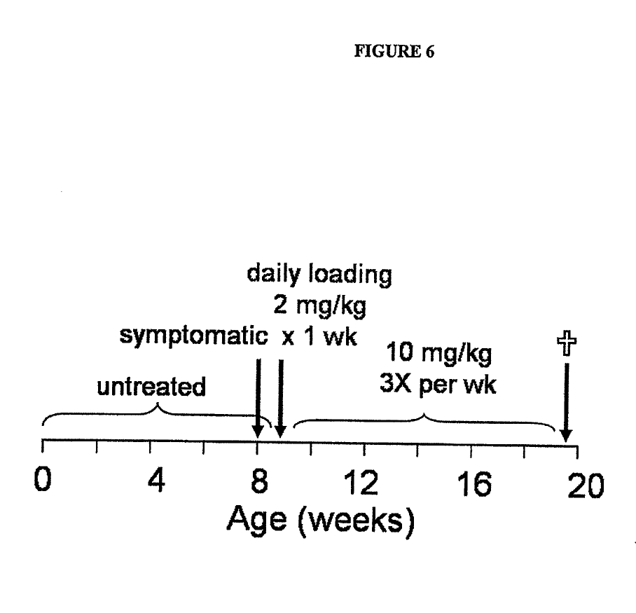

Studies were performed to determine if inhibition of NF-κB activation ameliorates accelerated aging in Ercc1^{-/Δ} mice. An Ercc1^{-/Δ} mouse was chronically treated with 8K-NBD peptide as described in FIG. 6. The age at onset of numerous progeroid symptoms were recorded (Table II). Ataxia and priapism, both caused by neurodegeneration (Rolig and McKinnon, 2000), were delayed by 3 wks and 1.5 wks respectively in the mouse treated with 8K-NBD. The mouse treated with vehicle only displayed cachexia, loss of muscle mass and vision by 16 wks of age. These symptoms did not arise in the sibling mouse treated with 8K-NBD within the 19 wk period of the study. This ≧3 wk delay in onset of symptoms suggests the possibility of extending quality of life for at least 15% of an organism's lifespan via inhibition of NF-κB activation.

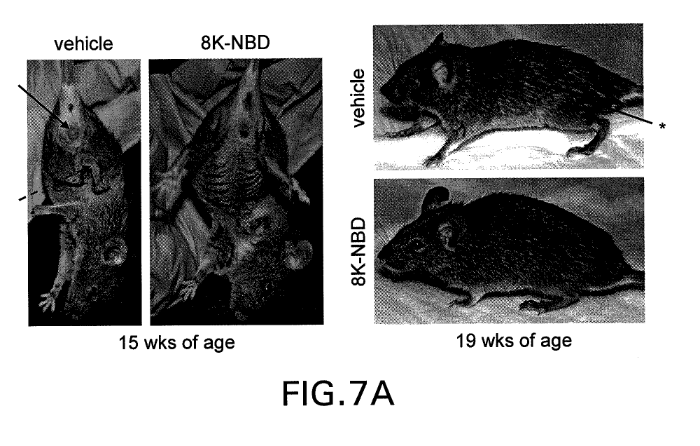

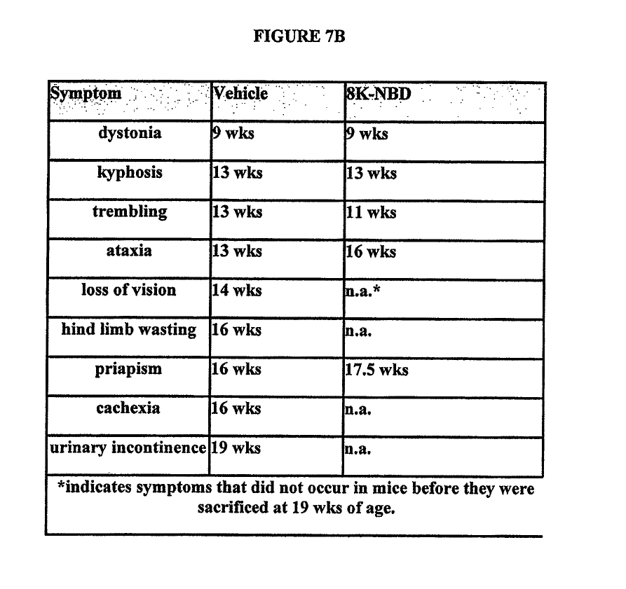

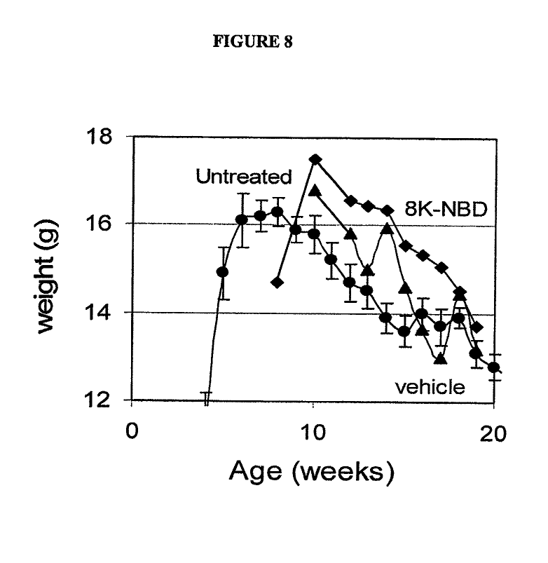

Although dystonia, trembling and kyphosis occurred in both mice, the severity of symptoms was significantly reduced in the mouse treated with 8K-NBD (FIG. 7). In addition, the mouse treated with 8K-NBD had improved ambulation and general appearance and maintained weight longer (FIG. 8).

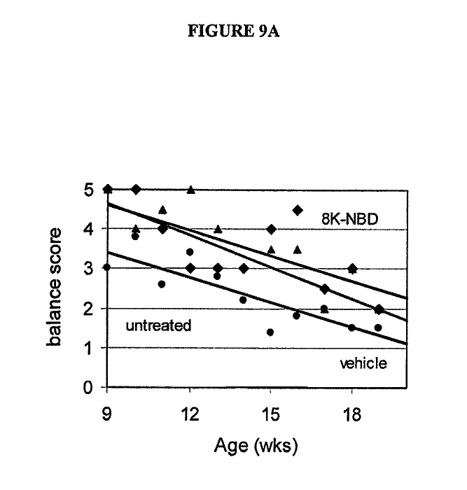

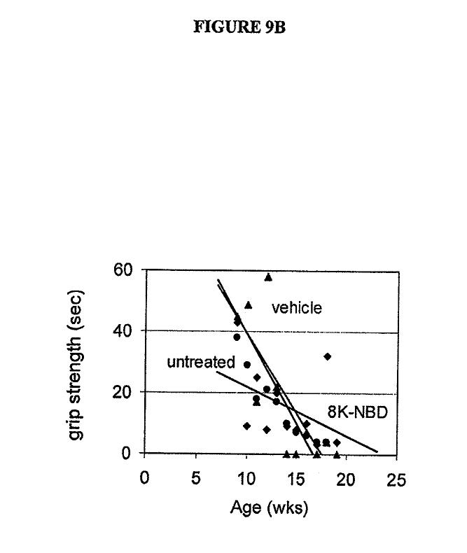

Motor strength and coordination were measured weekly in the mice by testing their ability to cross a balance beam (FIG. 9A) and to remain suspended from a wire (FIG. 9B). The mouse treated with 8K-NBD tended to perform better in both tests than untreated mice (n=7). These preliminary data demonstrate that the 8K-NBD peptide administered chronically at 10 mg/kg has a potent biological effect and supports the hypothesis that inhibition of NF-κB signaling can improve numerous debilitating symptoms that impair quality of life in a mouse model of accelerated aging.

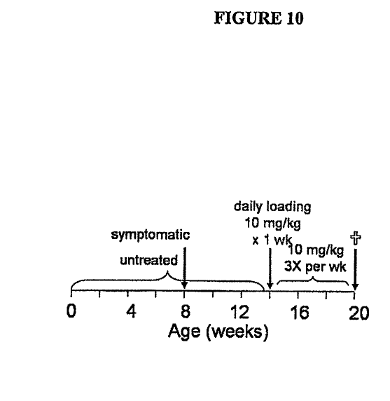

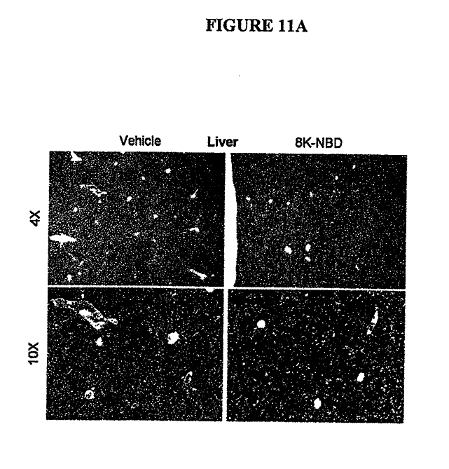

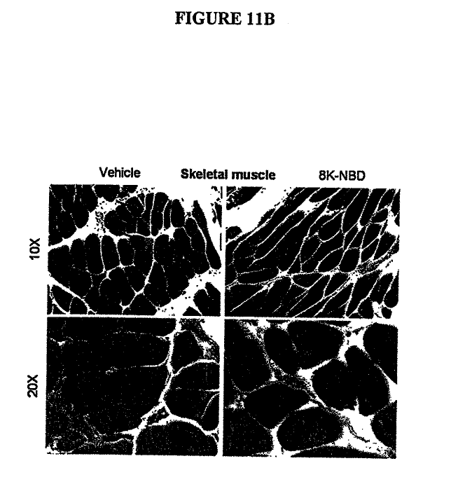

In a second series of experiments, an older Ercc1^{-/Δ} mouse, which already had progeroid symptoms, was chronically exposed to 8K-NBD then euthanized at 20 wk of age to determine if 8K-NBD affected histopathologic changes associated with aging (FIG. 10). Liver and skeletal muscle (SKM) were dissected, fixed, sectioned and stained. Tissues were compared to age-matched untreated Ercc1^{-/Δ}. The liver of Ercc1^{-/Δ} mice display large polyploid nuclei (FIG. 11A, 10×) characteristic of senescent hepatocytes (Gupta, 2000). In contrast, hepatocytes from a mouse treated with 8K-NBD showed a much higher cytoplasm to nuclear ratio. Notably, hepatic architecture was effaced in the liver of the 8K-NBD treated mouse, suggestive of liver regeneration (Michalopoulos and DeFrances, 2005). SKM of Ercc1^{-/Δ} mice showed evidence of necrosis (FIG. 11B). In contrast, SKM from the mouse treated with 8K-NBD is devoid of pathologic changes. This suggests that inhibition of NF-κB activation with 8K-NBD prevents degenerative changes in SKM. This is consistent with previous reports that in mouse models of numerous inflammatory myopathies, persistent NF-κB activity is associated with necrotic and regenerative changes (Monici et al., 2003).

In a third series of experiments, a different treatment regimen was used (FIG. 12) in which treatment with 8K-NBD or a negative control peptide, 8K-mNBD (having a mutated sequence, KKKKKKKKGGTALDASALQTE (SEQ ID NO:7)) was initiated earlier than the above regimens at approximately 5 weeks (8K-NBD or 8K-mNBD, 10 mg/kg) administered intraperitoneally three times per week). This was a double blinded twin study designed to eliminate genetic and environmental variables.

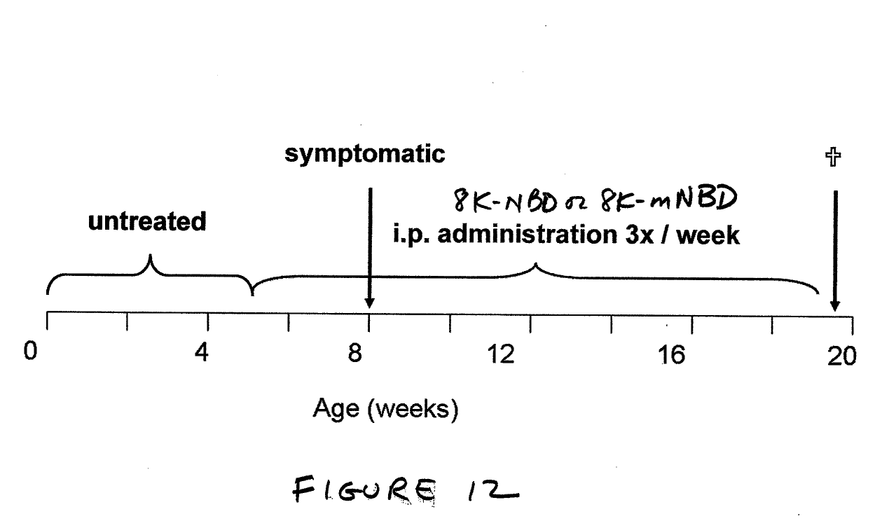

The results of this third series of experiments are shown in FIGS. 13-16. FIG. 13 presents a table showing the phenotypic changes associated with aging in mice treated with 8k-NBD or negative control 8k-mNBD. The overall aging score was improved by 30 percent in 8K-NBD versus 8K-mNBD-treated mice, indicating a significant delay in the onset of age-related symptoms corresponding to over a decade in human life.

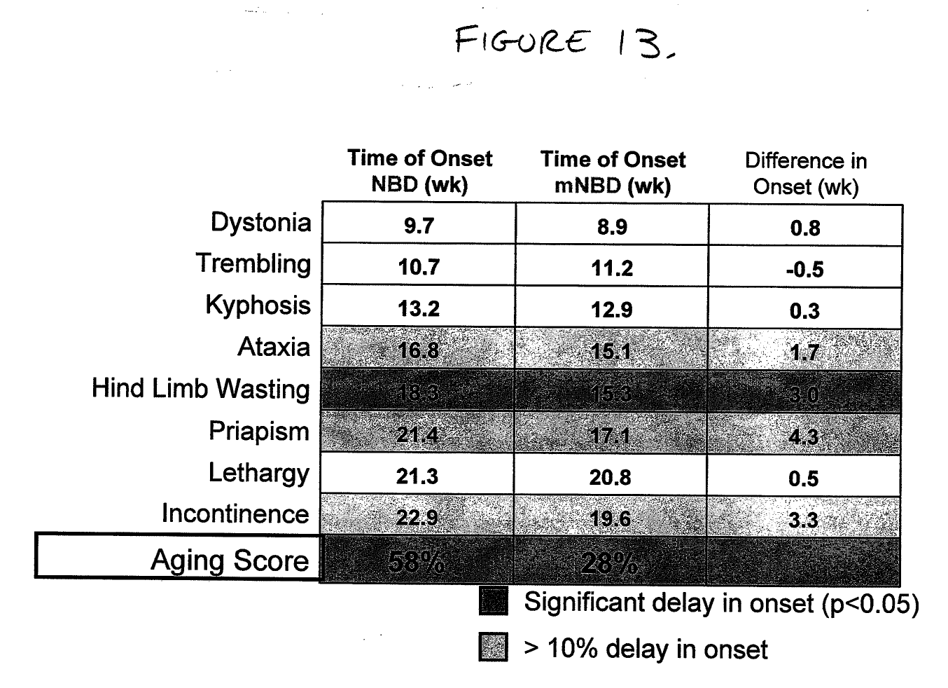

FIG. 14A-D shows a comparison of levels of neurodegeneration, as determined by glial fibrillary acidic protein (GFAP) staining, in young and senescent (aged, 2 yr) wild-type mice as well as Ercc1^{-/Δ} mice treated with either 8K-NBD or negative control 8K-mNBD according to the regimen of FIG. 12 at the age of 18 weeks. The GFAP staining pattern of the Ercc1^{-/Δ} mouse treated with 8K-NBD more closely resembles that of the young wild-type mouse relative to the 8K-mNBD-treated animal.

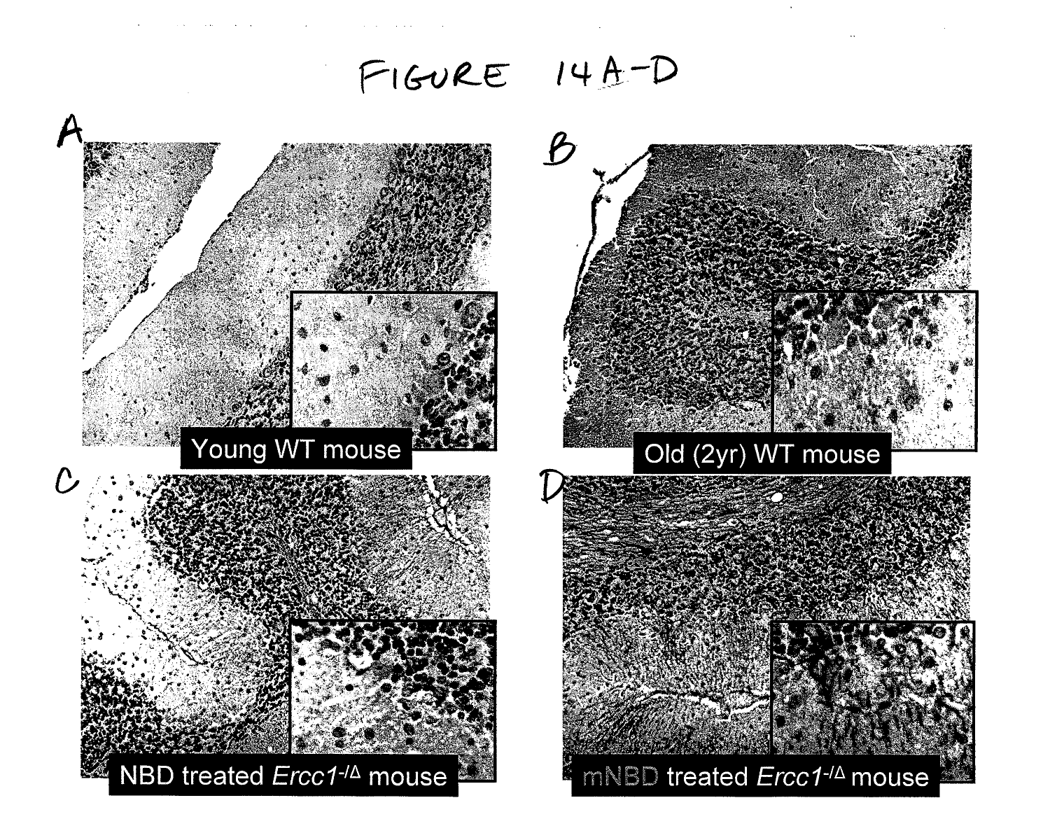

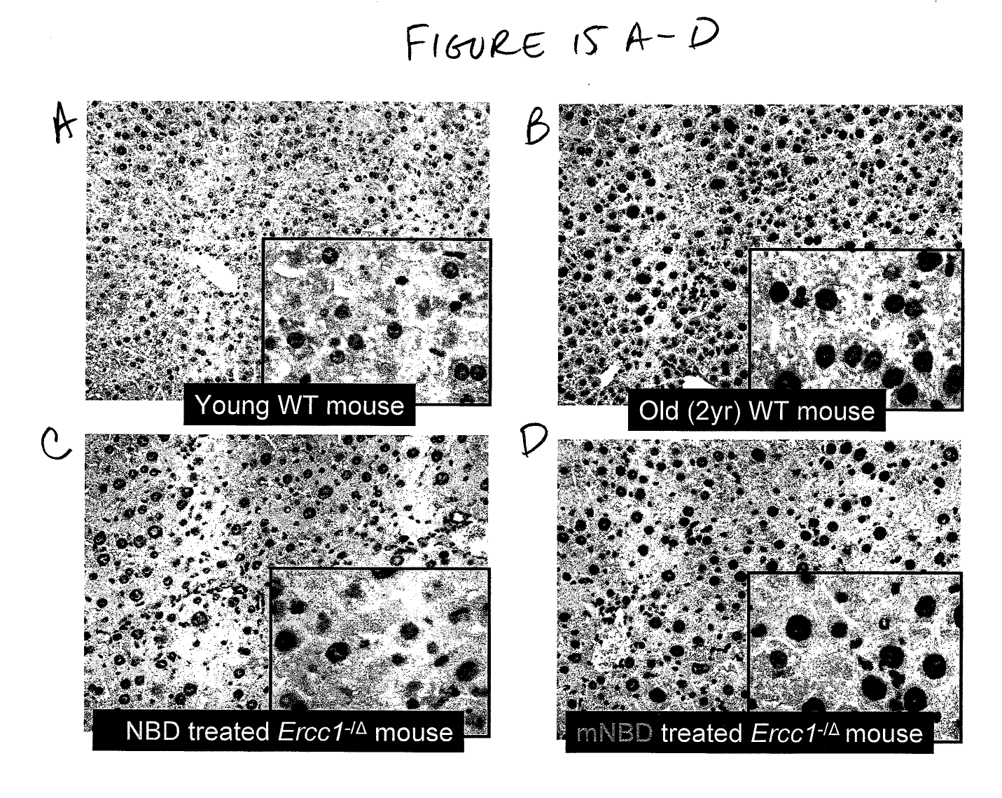

FIG. 15A-D presents a comparison of levels of hepatocellular senescence, as measured by staining for p16 protein, in young and senescent wild-type mice as well as Ercc1^{-/Δ} mice treated with either 8K-NBD or negative control 8K-mNBD according to the regimen of FIG. 12 at the age of 18 weeks. The staining pattern of the 8K-mNBD treated mouse more closely resembles the pattern in senescent wild-type hepatocytes, whereas the 8K-NBD-treated animal's staining pattern more closely resembles that of a young, healthy mouse.

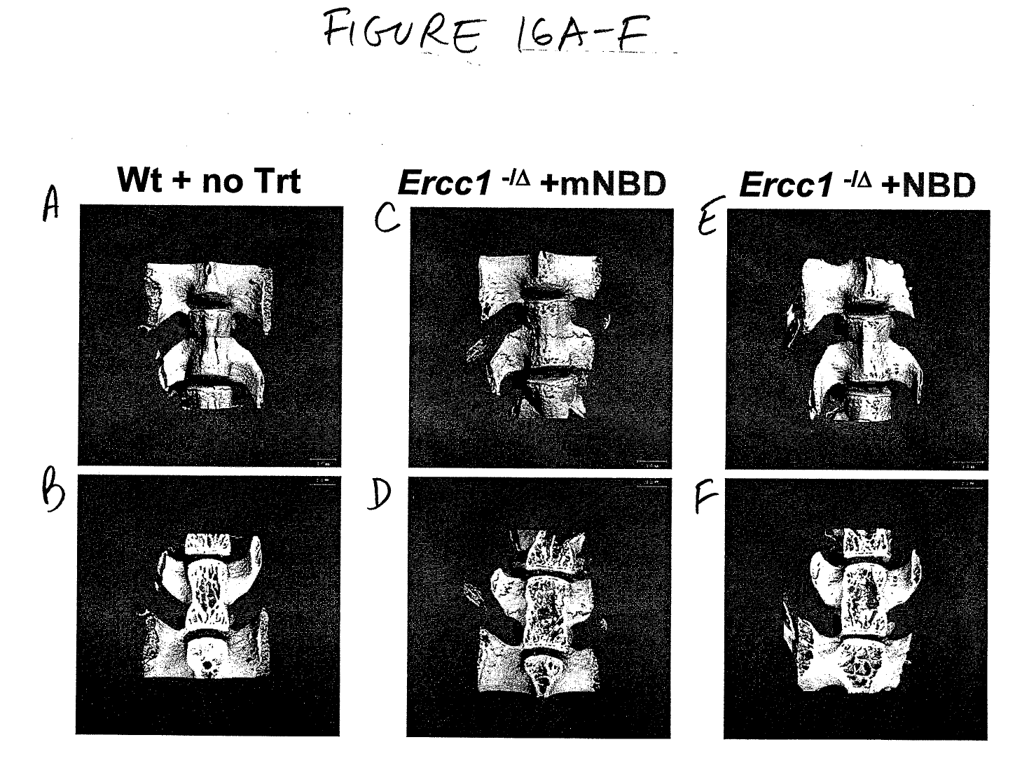

FIG. 16A-F shows a comparison of osteoporotic changes in Ercc1^{-/Δ} mice treated with 8K-NBD or negative control 8K-mNBD and a wild-type littermate at 17 weeks of age. The degree of osteoporotic changes appears greatest in the 8K-mNBD-treated animal.

In summary, these data indicate that NF-κB is activated in Ercc1^{-/Δ} mice, a mouse model that based on prior precedent (Niedernhofer et al., 2006) ages rapidly as a consequence of defective DNA repair. The foregoing data demonstrates that inhibition of NF-κB/IKK activation delays the pathogenic signs associated with aging, decreasing neurodegeneration, reducing cellular senescence in the liver, and delaying osteoporotic changes. This indicates that inhibition of NF-κB signaling in response to cellular stress may improve quality of life.

### 9. EXAMPLE

### Genetic Depletion of NF-κB

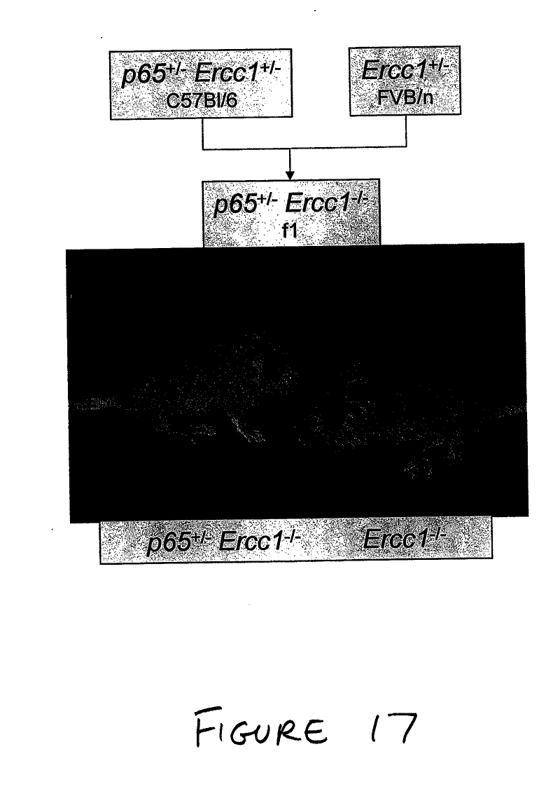

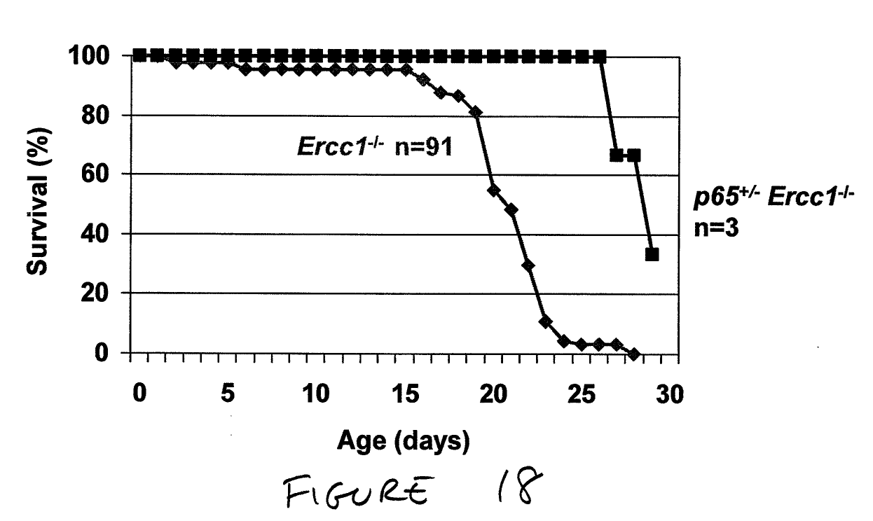

Mice genetically depleted in NF-κB were produced by crossing a p65^{+/-} Ercc^{+/-} parent with a Ercc^{+/-} parent and producing p65^{+/-} Ercc^{-/-} f1 progeny (FIG. 17). When the survival of these mice was studied, it was found that the median, although not the maximum, life span was increased in the p65^{+/-} Ercc^{-/-} mice (FIG. 18). This supports the conclusion that reduction in the level of NF-κB activity improves the quality of life, delaying the onset of age-related degenerative changes that limit life in individual members of the species, without significantly extending the lifespan of that species.

### 10. EXAMPLE

### PTD Transduction Through Skin

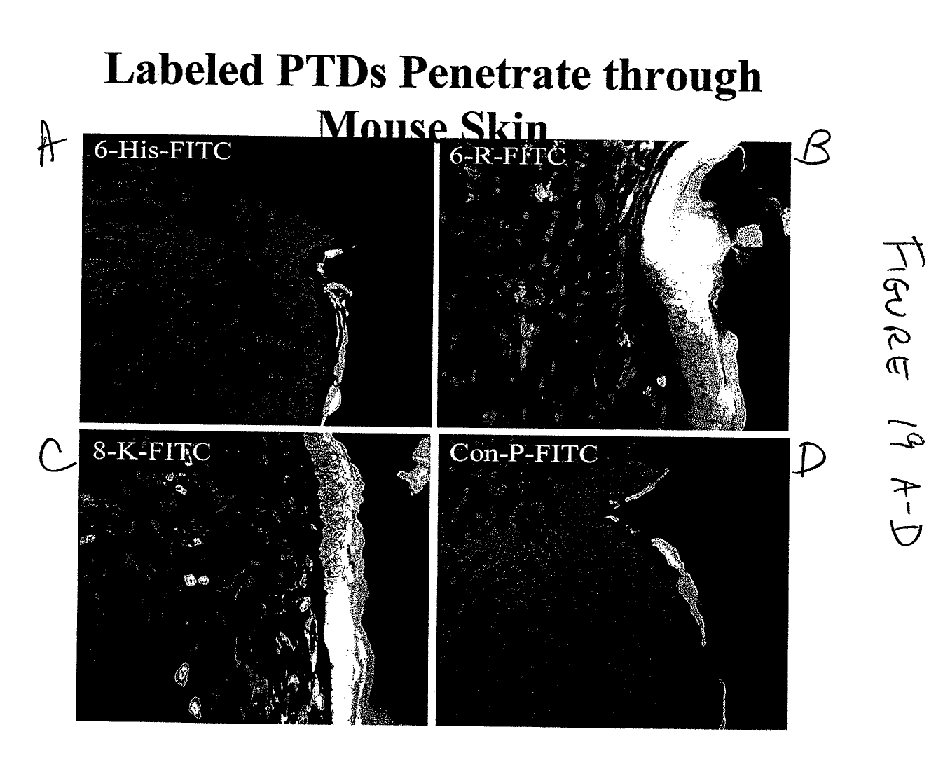

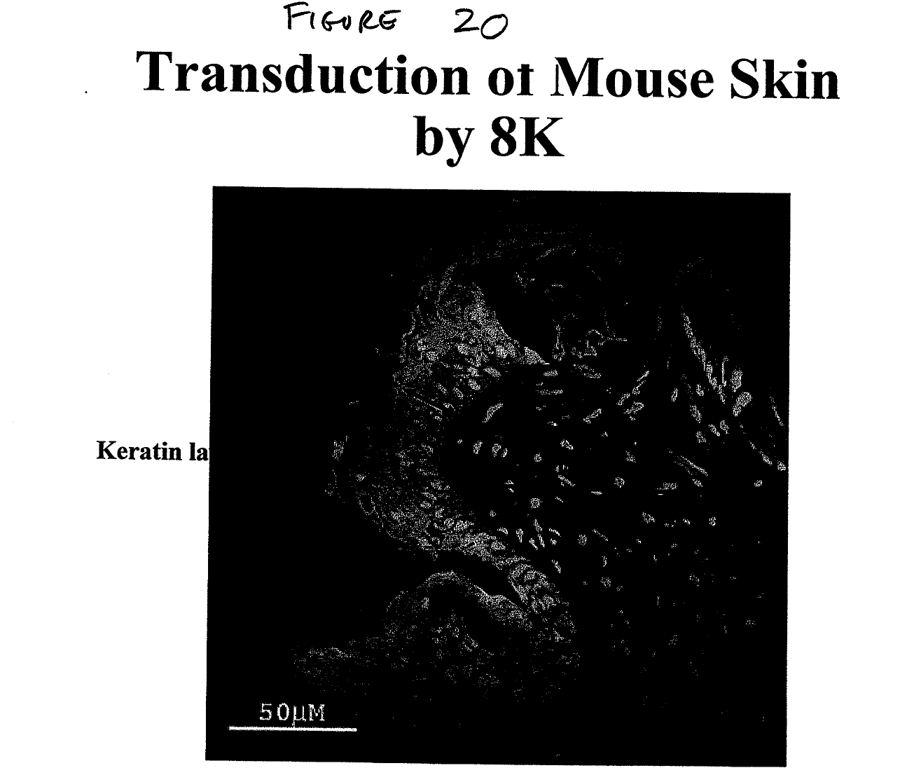

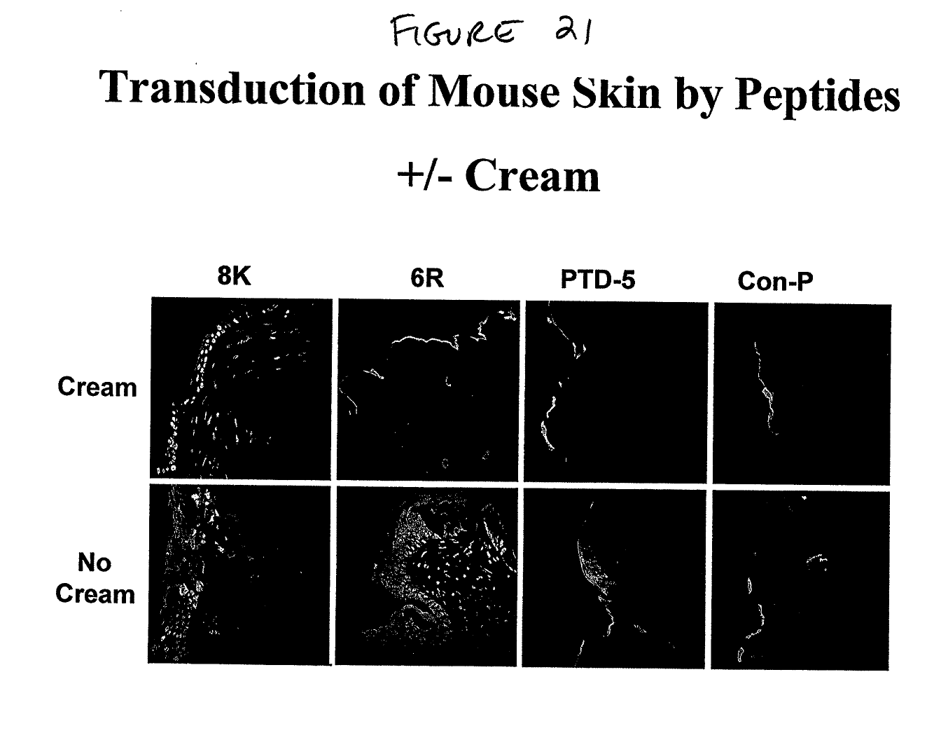

With regard to the applicability of the invention to topical administration of NF-κB activation inhibitors, experiments have been performed which demonstrate PTD transduction of mouse skin (see FIGS. 19A-D, 20 and 21). For these experiments, an equal volume of a standard base cream was mixed with the indicated FITC-labeled PTDs (5 mM), then applied to hairless (nude) mice for 3 hr. The skin was then isolated for confocal microscopy. For the experiment comparing transduction with or without cream, the PTDs were either applied (i) in a cream or (ii) in PBS (and covered with a bandaid to prevent drying).

### 11. EXAMPLE

### Delayed onset of frailty in an Ercc^{-/Δ} mouse treated with 5 MG/KG of Compound A

| | Oil | n | Compound A | n |
|---|---|---|---|---|
| Dystonia | 6.8 | 3 | 13.7 | 1 |
| Trembling | 8.5 | 3 | 12.7 | 1 |
| Kyphosis | 10.8 | 3 | 14.3 | 1 |
| Ataxia | 13.2 | 3 | 19.3 | 1 |
| Wasting | 14.2 | 3 | 19.7 | 1 |

### 12. REFERENCES

Bao, J. J., Lee, B. P., Stephens, L. C., Sahin, A. A., Van, N. T., Johnston, D. A., Ou, C. N. and Kuo, M. T. (2000) Elevated expression of hepatic proliferative markers during early hepatocarcinogenesis in hepatitis-B virus transgenic mice lacking mdrla-encoded P-glycoprotein. Mol Carcinog, 29, 103-111.
Bartke, A. (2005) Minireview: role of the growth hormone/insulin-like growth factor system in mammalian aging. Endocrinology, 146, 3718-3723.
Bender, K., Gottlicher, M., Whiteside, S., Rahmsdorf, H. J. and Herrlich, P. (1998) Sequential DNA damage-independent and -dependent activation of NF-kappaB by UV. Embo J, 17, 5170-5181.
Bregegere, F., Milner, Y. and Friguet, B. (2006) The ubiquitin-proteasome system at the crossroads of stress-response and ageing pathways: a handle for skin care? Ageing Res Rev, 5, 60-90.
Brooks, P. J. (2002) DNA repair in neural cells: basic science and clinical implications. Mutat Res, 509, 93-108.
Browne, S. E., Roberts, L. J., 2nd, Dennery, P. A., Doctrow, S. R., Beal, M. F., Barlow, C. and Levine, R. L. (2004) Treatment with a catalytic antioxidant corrects the neurobehavioral defect in ataxia-telangiectasia mice. Free Radic Biol Med, 36, 938-942.
Chipchase, M. D., O'Neill, M. and Melton, D. W. (2003) Characterization of premature liver polyploidy in DNA repair (Ercc1)-deficient mice. Hepatology, 38, 958-966.
Crawley, J. N. (2000) What's wrong with my mouse? Wiley-Liss, New York.
Crippen, D. L. (2000) Preparing for an Aging Population. Committee on the Budget, U.S. House of Representatives, Washington, D. C.
Dai, S., Hirayama, T., Abbas, S. and Abu-Amer, Y. (2004) The IkappaB kinase (IKK) inhibitor, NEMO-binding domain peptide, blocks osteoclastogenesis and bone erosion in inflammatory arthritis. J Biol Chem, 279, 37219-37222.
Dasgupta, S., Jana, M., Zhou, Y., Fung, Y. K., Ghosh, S. and Pahan, K. (2004) Antineuroinflammatory effect of NF-kappaB essential modifier-binding domain peptides in the adoptive transfer model of experimental allergic encephalomyelitis. J Immunol, 173, 1344-1354.
Davé, S. H., Tilstra, J. S., Li, F., Matsuoka, K., Sepulveda, A. R., Robbins, P. D. and Plevy, S. E. (2007) Amelioration of chronic murine colitis by peptide mediated transduction of the IkB kinase (IKK) inhibitor NEMO binding domain (NBD) peptide. Journal of Immunology, 179, 7852-7859.
De Bosscher et al. (2005) Proc. Natl. Acad. Sci. U.S.A., 102, 15827-15832.
de Boer, J., Andressoo, J. O., de Wit, J., Huijmans, J., Beems, R. B., van Steeg, H., Weeda, G., van der Horst, G. T., van Leeuwen, W., Themmen, A. P., Meradji, M. and Hoeijmakers, J. H. (2002) Premature aging in mice deficient in DNA repair and transcription. Science, 296, 1276-1279.
di Meglio, P., lanaro, A. and Ghosh, S. (2005) Amelioration of acute inflammation by systemic administration of a cell-permeable peptide inhibitor of NF-kappaB activation. Arthritis Rheum, 52, 951-958.
Dolle, M. E., Snyder, W. K., Dunson, D. B. and Vijg, J. (2002) Mutational fingerprints of aging. Nucleic Acids Res, 30, 545-549.
Ferguson, V. L., Ayers, R. A., Bateman, T. A. and Simske, S. J. (2003) Bone development and age-related bone loss in male C57BL/6J mice. Bone, 33, 387-398.
Giardina, C. and Hubbard, A. K. (2002) Growing old with nuclear factor-kappaB. Cell Stress Chaperones, 7, 207-212.
Go, E. K., Jung, K. J., Kim, J. Y., Yu, B. P. and Chung, H. Y. (2005) Betaine suppresses proinflammatory signaling during aging: the involvement of nuclear factor-κB via nuclear factor-inducing kinase/IκB kinase and mitogen-activated protein kinases. J. Gerontol, 60A, 1252-1264.
Gosselin, K. and Abbadie, C. (2003) Involvement of Rel/NF-kappa B transcription factors in senescence. Exp Gerontol, 38, 1271-1283.
Gupta, S. (2000) Hepatic polyploidy and liver growth control. Semin Cancer Biol, 10, 161-171.
Habraken, Y. and Piette, J. (2006) NF-kappaB activation by double-strand breaks. Biochem Pharmacol, 72, 1132-1141.
Hamilton, M. L., Van Remmen, H., Drake, J. A., Yang, H., Guo, Z. M., Kewitt, K., Walter, C. A. and Richardson, A. (2001) Does oxidative damage to DNA increase with age? Proc Natl Acad Sci USA, 98, 10469-10474.
Hasty, P., Campisi, J., Hoeijmakers, J., van Steeg, H. and Vijg, J. (2003) Aging and genome maintenance: lessons from the mouse? Science, 299, 1355-1359.
Hekimi, S. and Guarente, L. (2003) Genetics and the specificity of the aging process. Science, 299, 1351-1354.
Helenius, M., Hanninen, M., Lehtinen, S. K. and Salminen, A. (1996a) Aging-induced up-regulation of nuclear binding activities of oxidative stress responsive NF-κB transcription factor in mouse cardiac muscle. J Mol Cell Cardiol, 28, 487-498.
Helenius, M., Hanninen, M., Lehtinen, S. K. and Salminen, A. (1996b) Changes associated with aging and replicative senescence in the regulation of transcription factor nuclear factor-kappa B. Biochem J, 318 (Pt 2), 603-608.
Helenius, M., Kyrylenko, S., Vehvilainen, P. and Salminen, A. (2001) Characterization of aging-associated up-regulation of constitutive nuclear factor-kappa B binding activity. Antioxid Redox Signal, 3, 147-156.
Hu, M. C. and Hung, M. C. (2005) Role of IkappaB kinase in tumorigenesis. Future Oncol, 1, 67-78.
Janssens, S. and Tschopp, J. (2006) Signals from within: the DNA-damage-induced NF-kappaB response. Cell Death Differ, 13, 773-784.
Jimi, E., Aoki, K., Saito, H., D'Acquisto, F., May, M. J., Nakamura, I., Sudo, T., Kojima, T., Okamoto, F., Fukushima, H., Okabe, K., Ohya, K. and Ghosh, S. (2004) Selective inhibition of NF-kappa B blocks osteoclastogenesis and prevents inflammatory bone destruction in vivo. Nat Med, 10, 617-624.
Keller, H. L., St Pierre Schneider, B., Eppihimer, L. A. and Cannon, J. G. (1999) Association of IGF-I and IGF-II with myofiber regeneration in vivo. Muscle Nerve, 22, 347-354.
Kenyon, C. (2005) The plasticity of aging: insights from long-lived mutants. Cell, 120, 449-460.
Kim, J. Y., Jung, K. J., Choi, J. S., and Chung, H. Y. (2006) Modulation of age-related nuclear factor-KB (NF-κB) pathway by hesperetin. Aging Cell, 5, 401-411.
Kirkwood, T. B. (2005a) Time of our lives. What controls the length of life? EMBO Rep, 6 Spec No, S4-8.
Kirkwood, T. B. (2005b) Understanding the odd science of aging. Cell, 120, 437-447.
Korhonen, P., Helenius, M. and Salminen, A. (1997) Age-related changes in the regulation of transcription factor NF-kappa B in rat brain. Neurosci Lett, 225, 61-64.
Kovalovich, K., DeAngelis, R. A., Li, W., Furth, E. E., Ciliberto, G. and Taub, R. (2000) Increased toxin-induced liver injury and fibrosis in interleukin-6-deficient mice. Hepatology, 31, 149-159.
Kurosu, H., Yamamoto, M., Clark, J. D., Pastor, J. V., Nandi, A., Gumani, P., McGuinness, O. P., Chikuda, H., Yamaguchi, M., Kawaguchi, H., Shimomura, I., Takayama, Y., Herz, J., Kahn, C. R., Rosenblatt, K. P. and Kuro-o, M. (2005) Suppression of aging in mice by the hormone Klotho. Science, 309, 1829-1833.
Lantinga-van Leeuwen, I. S., Dauwerse, J. G., Baelde, H. J., Leonhard, W. N., van de Wal, A., Ward, C. J., Verbeek, S., Deruiter, M. C., Breuning, M. H., de Heer, E. and Peters, D. J. (2004) Lowering of Pkd1 expression is sufficient to cause polycystic kidney disease. Hum Mol Genet, 13, 3069-3077.
Lee, J. H., Koo, T. H., Yoon, H., Jung, H. S., Jin, H. Z., Lee, K., Hong, Y. S., Lee, J. J (2006) Inhibition of NF-kappa B activation through targeting I kappa B kinase by celastrol, a quinone methide triterpenoid. Biochem Pharmacol 72(10) 1311-21.
Li, Q., Van Antwerp, D., Mercurio, F., Lee, K. F. and Verma, I. M. (1999) Severe liver degeneration in mice lacking the IkappaB kinase 2 gene. Science, 284, 321-325.
Liou, Y. C., Sun, A., Ryo, A., Zhou, X. Z., Yu, Z. X., Huang, H. K., Uchida, T., Bronson, R., Bing, G., Li, X., Hunter, T. and Lu, K. P. (2003) Role of the prolyl isomerase Pin1 in protecting against age-dependent neurodegeneration. Nature, 424, 556-561.
Louw et al. (1997) Biochem. Pharmacol., 53, 189-197.
Louw et al. (1999) Endocrinol., 140, 2044-2053.
Maier, B., Gluba, W., Bernier, B., Turner, T., Mohammad, K., Guise, T., Sutherland, A., Thorner, M. and Scrable, H. (2004) Modulation of mammalian life span by the short isoform of p53. Genes Dev, 18, 306-319.
May, M. J., D'Acquisto, F., Madge, L. A., Glockner, J., Pober, J. S. and Ghosh, S. (2000) Selective inhibition of NF-kappaB activation by a peptide that blocks the interaction of NEMO with the IkappaB kinase complex. Science, 289, 1550-1554.
May, M. J., Marienfeld, R. B. and Ghosh, S. (2002) Characterization of the Ikappa B-kinase NEMO binding domain. J Biol Chem, 277, 45992-46000.
McWhir, J., Selfridge, J., Harrison, D. J., Squires, S. and Melton, D. W. (1993) Mice with DNA repair gene (ERCC-1) deficiency have elevated levels of p53, liver nuclear abnormalities and die before weaning. Nat Genet, 5, 217-224.
Mi, Z., Mai, J., Lu, X. and Robbins, P. D. (2000) Characterization of a class of cationic peptides able to facilitate efficient protein transduction in vitro and in vivo. Mol Ther, 2, 339-347.
Michalopoulos, G. K. and DeFrances, M. (2005) Liver regeneration. Adv Biochem Eng Biotechnol, 93, 101-134.
Migliaccio, E., Giorgio, M., Mele, S., Pelicci, G., Reboldi, P., Pandolfi, P. P., Lanfrancone, L. and Pelicci, P. G. (1999) The p66shc adaptor protein controls oxidative stress response and life span in mammals. Nature, 402, 309-313.
Monici, M. C., Aguennouz, M., Mazzeo, A., Messina, C. and Vita, G. (2003) Activation of nuclear factor-kappaB in inflammatory myopathies and Duchenne muscular dystrophy. Neurology, 60, 993-997.
Murata, T., Shimada, M., Sakakibara, S., Yoshino, T., Kadono, H., Masuda, T., Shimazaki, M., Shintani, T., Fuchikami, K., Sakai, K., Inbe, H., Takeshita, K., Niki, T., Umeda, M., Bacon, K. B., Ziegelbauer, K. B. and Lowinger, T. B. (2003) Discovery of novel and selective IKK-beta serine-threonine protein kinase inhibitors. Part 1. Bioorg Med Chem Lett, 13, 913-918.
Mustafa, I. and Leverve, X. (2001) Metabolic and nutritional disorders in cardiac cachexia. Nutrition, 17, 756-760.
Nenci, A., Becker, C., Wullaert, A., Gareus, R., van Loo, G., Danese, S., Huth, M., Nikolaev, A., Neufert, C., Madison, B., Gumucio, D., Neurath, M. F. and Pasparakis, M. (2007) Epithelial NEMO links innate immunity to chronic intestinal inflammation. Nature, 446, 557-561.
Niedernhofer, L. J., Garinis, G. A., Raams, A., Lalai, A. S., Robinson, A. R., Appeldoom, E., Odijk, H., Oostendorp, R., Ahmad, A., van Leeuwen, W., Theil, A. F., Vermeulen, W., van der Horst, G. T., Meinecke, P., Kleijer, W. J., Vijg, J., Jaspers, N. G. and Hoeijmakers, J. H. (2006) A new progeroid syndrome reveals that genotoxic stress suppresses the somatotroph axis. Nature, 444, 1038-1043.
Niedernhofer, L. J., Odijk, H., Budzowska, M., van Drunen, E., Maas, A., Theil, A. F., de Wit, J., Jaspers, N. G., Beverloo, H. B., Hoeijmakers, J. H. and Kanaar, R. (2004) The structure-specific endonuclease Ercc1-Xpf is required to resolve DNA interstrand cross-link-induced double-strand breaks. Mol Cell Biol, 24, 5776-5787.
Park, K. C., Park, E. J., Kim, E. R., Kim, Y., Chung, S. H., Cho, B. W., Kim, S., Jin, M. (2005) Therapeutic effects of PG201, an ethanol extract from herbs, through cartilage protection on collagenase-induced arthritis in rabbits. Biochem Biophys Res Commun 331(4) 1469-77.
Parrinello, S., Samper, E., Krtolica, A., Goldstein, J., Melov, S. and Campisi, J. (2003) Oxygen sensitivity severely limits the replicative lifespan of murine fibroblasts. Nat Cell Biol, 5, 741-747.
Pikarsky, E., Porat, R. M., Stein, I., Abramovitch, R., Amit, S., Kasem, S., Gutkovich-Pyest, E., Urieli-Shoval, S., Galun, E. and Ben-Neriah, Y. (2004) NF-kappaB functions as a tumour promoter in inflammation-associated cancer. Nature, 431, 461-466.
Prasher, J. M., Lalai, A. S., Heijmans-Antonissen, C., Ploemacher, R. E., Hoeijmakers, J. H., Touw, I. P. and Niedernhofer, L. J. (2005) Reduced hematopoietic reserves in DNA interstrand crosslink repair-deficient Ercc1−/− mice. Embo J, 24, 861-871.
Resnick, N. M. and Dosa, D. (2004) Geriatric Medicine. In Kasper, D. L., Braunwald, E., Fauci, A. S., Hauser, S. L., Longo, D. L. and Jameson, J. L. (eds.), Harrison's Principles of Internal Medicine. McGraw-Hill, Montreal, pp. 249-259.
Rolig, R. L. and McKinnon, P. J. (2000) Linking DNA damage and neurodegeneration. Trends Neurosci, 23, 417-424.
Roubenoff, R. (1999) The pathophysiology of wasting in the elderly. J Nutr, 129, 256S-259S.
Rowlatt, C., Chesterman, F. C. and Sheriff, M. U. (1976) Lifespan, age changes and tumour incidence in an ageing C57BL mouse colony. Lab Anim, 10, 419-442.
Rudolph, D., Yeh, W. C., Wakeham, A., Rudolph, B., Nallainathan, D., Potter, J., Elia, A. J. and Mak, T. W. (2000) Severe liver degeneration and lack of NF-kappaB activation in NEMO/IKKgamma-deficient mice. Genes Dev, 14, 854-862.
Selfridge, J., Hsia, K. T., Redhead, N. J. and Melton, D. W. (2001) Correction of liver dysfunction in DNA repair-deficient mice with an ERCC1 transgene. Nucleic Acids Res, 29, 4541-4550.
Sijbers, A. M., de Laat, W. L., Ariza, R. R., Biggerstaff, M., Wei, Y. F., Moggs, J. G., Carter, K. C., Shell, B. K., Evans, E., de Jong, M. C., Rademakers, S., de Rooij, J., Jaspers, N. G., Hoeijmakers, J. H. and Wood, R. D. (1996) Xeroderma pigmentosum group F caused by a defect in a structure-specific DNA repair endonuclease. Cell, 86, 811-822.
Tak, P. P. and Firestein, G. S. (2001) NF-kappaB: a key role in inflammatory diseases. J Clin Invest, 107, 7-11.
Tanaka, M., Fuentes, M. E., Yamaguchi, K., Durnin, M. H., Dalrymple, S. A., Hardy, K. L. and Goeddel, D. V. (1999) Embryonic lethality, liver degeneration, and impaired NF-kappa B activation in IKK-beta-deficient mice. Immunity, 10, 421-429.
te Poele, R. H., Okorokov, A. L., Jardine, L., Cummings, J. and Joel, S. P. (2002) DNA damage is able to induce senescence in tumor cells in vitro and in vivo. Cancer Res, 62, 1876-1883.
Tian, M., Shinkura, R., Shinkura, N. and Alt, F. W. (2004) Growth retardation, early death, and DNA repair defects in mice deficient for the nucleotide excision repair enzyme XPF. Mol Cell Biol, 24, 1200-1205.
U.S. Department of Health and Human Services, A.o.A. (2003) A profile of older Americans: 2003.
van der Pluijm, I., Garinis, G. A., Brandt, R. M., Gorgels, T. G., Wijnhoven, S. W., Diderich, K. E., de Wit, J., Mitchell, J. R., van Oostrom, C., Beems, R., Niedernhofer, L. J., Velasco, S., Friedberg, E. C., Tanaka, K., van Steeg, H., Hoeijmakers, J. H. and van der Horst, G. T. (2007) Impaired genome maintenance suppresses the growth hormone—insulin-like growth factor 1 axis in mice with Cockayne syndrome. PLoS Biol, 5, e2.
Wadia, J. S. and Dowdy, S. F. (2002) Protein transduction technology. Curr Opin Biotechnol, 13, 52-56.
Weeda, G., Donker, I., de Wit, J., Morreau, H., Janssens, R., Vissers, C. J., Nigg, A., van Steeg, H., Bootsma, D. and Hoeijmakers, J. H. (1997) Disruption of mouse ERCC1 results in a novel repair syndrome with growth failure, nuclear abnormalities and senescence. Curr Biol, 7, 427-439.
Xiao, Z. Q. and Majumdar, A. P. (2000) Induction of transcriptional activity of AP-1 and NF-kappaB in the gastric mucosa during aging. Am J Physiol Gastrointest Liver Physiol, 278, G855-865.
Ziegelbauer, K., Gantner, F., Lukacs, N. W., Berlin, A., Fuchikami, K., Niki, T., Sakai, K., Inbe, H., Takeshita, K., Ishimori, M., Komura, H., Murata, T., Lowinger, T. and Bacon, K. B. (2005) A selective novel low-molecular-weight inhibitor of IkappaB kinase-beta (IKK-beta) prevents pulmonary inflammation and shows broad anti-inflammatory activity. Br J Pharmacol, 145, 178-192.
Wen, D., Nong, Y., Morgan, J. G., Gangurde, P., Bielecki, A., Dasilva, J., Keaveney, M., Cheng, H., Fraser, C., Schopf, L., Hepperle, M., Harriman, G., Jaffee, B. D., Ocain, T. D., Xu, Y. (2006) A selective small molecule IkappaB Kinase beta inhibitor blocks nuclear factor kappaB-mediated inflammatory responses in human fibroblast-like synoviocytes, chondrocytes, and mast cells. J Pharmacol Exp Ther 317(3), 989-1001.
Kishore, N., Sommers, C., Mathialagan, S., Guzova, J., Yao, M., Hauser, S., Huynh, K., Bonar, S., Mielke, C., Albee, L., Weier, R., Graneto, M., Hanau, C.,Perry, T., Tripp, C. S. (2003) A selective IKK-2 inhibitor blocks NF-kappa B-dependent gene expression in interleukin-1 beta-stimulated synovial fibroblasts. J Biol Chem 278(35), 32861-71.

Various publications are cited herein, the contents of which are hereby incorporated by reference in their entireties.

What is claimed is:

1. A method of inhibiting one or more signs of aging in a subject in need of such treatment, comprising administering, to the subject, an effective amount of an inhibitor of NF-κB activation, wherein the sign of aging is selected from the group consisting of the development or progression of one or more signs of aging, including, but not limited to, epidermal atrophy, epidermal hyperpigmentation, wrinkles, hearing loss, visual impairment, cerebral atrophy, cognitive deficits, trembling, ataxia, cerebellar degeneration, hypertension, renal insufficiency, renal acidosis, incontinence, decreased liver function, hypoalbuminemia, hepatic accumulation of glycogen and triglycerides, anemia, bone marrow degeneration, osteopenia, kyphosis, degenerative joint disease, intervertebral disc degeneration, sarcopenia, muscle weakness, dystonia, increased peroxisome biogenesis, increased apoptosis, decreased cellular proliferation, cachexia, and decreased lifespan.

2. The method of claim 1, wherein the inhibitor of NF-κB activation is a NBD peptide.

3. The method of claim 1, wherein the inhibitor of NF-κB activation is selected from the group consisting of BAY11-7082, BAY11-7085, SC-514, MG132, TPCK, ML120B, PG201, Celastrol, and MLN0415.

4. The method of claim 1, wherein the inhibitor of NF-κB activation is a 2-amino-3-cyna-4-alkyl-6-(2-hydroxyphenyl)pyridine derivative.

5. The method of claim 4, wherein the 2-amino-3-cyna-4-alkyl-6-(2-hydroxyphenyl)pyridine derivative is selected from the group consisting of Compound A and Compound B.

6. A method of improving age-related performance in a geriatric subject, comprising administering, to the subject, an effective amount of an inhibitor of NF-κB activation.

7. The method of claim 6, wherein the inhibitor of NF-κB activation is a NBD peptide.

8. The method of claim 6, wherein the inhibitor of NF-κB activation is selected from the group consisting of BAY11-7082, BAY11-7085, SC-514, MG132, TPCK, ML120B, PG201, Celastrol, and MLN0415

9. The method of claim 6, wherein the inhibitor of NF-κB activation is a 2-amino-3-cyna-4-alkyl-6-(2-hydroxyphenyl)pyridine derivative.

10. The method of claim 9, wherein the 2-amino-3-cyna-4-alkyl-6-(2-hydroxyphenyl)pyridine derivative is selected from the group consisting of Compound A and Compound B.

11. A method of prolonging survival of a geriatric subject, comprising administering, to the subject, an effective amount of an inhibitor of NF-κB activation.

12. The method of claim 11, wherein the inhibitor of NF-κB activation is a NBD peptide.

13. The method of claim 11, wherein the inhibitor of NF-κB activation is selected from the group consisting of BAY11-7082, BAY11-7085, SC-514, MG132, TPCK, ML120B, PG201, Celastrol, and MLN0415

14. The method of claim 11, wherein the inhibitor of NF-κB activation is a 2-amino-3-cyna-4-alkyl-6-(2-hydroxyphenyl)pyridine derivative.

15. The method of claim 14, wherein the 2-amino-3-cyna-4-alkyl-6-(2-hydroxyphenyl)pyridine derivative is selected from the group consisting of Compound A and Compound B.

16. An isolated NBD peptide comprising (i) a polylysine transduction peptide of between four and twelve lysine residues and (ii) a NEMO binding domain with a sequence selected from the group consisting of (a) TALDWSWLQTE (SEQ ID NO:1), (b) a sequence which is at least 90 percent homologous to SEQ ID NO:1, (c) a 9-11 amino acid sequence which differs from SEQ ID NO:1 in no more than two amino acids, and (d) a 8-11 amino acid sequence which differs from SEQ ID NO:1 in no more than three amino acids.

17. The isolated NBD peptide of claim 16, wherein the polylysine transduction peptide consists of eight lysine residues.

18. A pharmaceutical composition comprising the NBD peptide of claim 16, together with a pharmaceutical carrier.

19. A pharmaceutical composition comprising the NBD peptide of claim 17, together with a pharmaceutical carrier.


SEQUENCE LISTING (7 sequences)

SEQ ID NO: 1 - protein, 11 aa, Artificial Sequence
  Synthetic peptide
TALDWSWLQTE

SEQ ID NO: 2 - protein, 21 aa, Artificial Sequence
  Synthetic peptide
KKKKKKKKGGTALDWSWLQTE

SEQ ID NO: 3 - protein, 6 aa, Artificial Sequence
  Synthetic peptide
HHHHHH

SEQ ID NO: 4 - protein, 6 aa, Artificial Sequence
  Synthetic peptide
RRRRRR

SEQ ID NO: 5 - protein, 8 aa, Artificial Sequence
  Synthetic peptide
KKKKKKKK

SEQ ID NO: 6 - protein, 12 aa, Artificial Sequence
  Synthetic peptide
ARFLEHGSDKAT

SEQ ID NO: 7 - protein, 21 aa, Artificial Sequence
  Synthetic peptide
KKKKKKKKGGTALDASALQTE